(19) United States
(12) Patent Application Publication
(10) Pub. No.: US 2004/0101577 A1
(43) Pub. Date: May 27, 2004

(54) Composition for treating acne vulgaris and fabrication method

(76) Inventors: II-Ho Ahn, Seoul (KR); Jong-In Kim, Seoul (KR); Soonsun Hong, Seoul (KR); Jong-Hyuk Sung, Chonrabuk-do (KR)

Correspondence Address: BROWDY AND NEIMARK, P.L.L.C., 624 NINTH STREET, NW, SUITE 300, WASHINGTON, DC, 20001-5303, US

(21) Appl. No.: 10/473,044

(22) PCT Filed: Oct. 16, 2001
(86) PCT No.: PCT/KR01/01740

(30) Foreign Application Priority Data
Mar. 27, 2001 (KR) 2001/15946
Apr. 10, 2001 (KR) 2001/18830

(51) Int. Cl. A61K 35/78; A61K 31/724

(52) U.S. Cl. 424/725; 424/757; 514/58; 424/764

(57) ABSTRACT

The present invention relates to the composition for treating acne vulgaris and fabrication method. To manufacture the composition, the present invention mixes appropriate quantities of burdock extract, glycyrrhiza extract, phellodendron extract, and salicylic acid. The present invention embodies the composition that individually corresponds and treats other causes such as “inflammation/germ”, which is known as a principal cause of acne occurrence. Moreover, the composition is stabilized with hydroxy beta cyclodextrin and silicon polymer, and is not affected as time for the purpose of treating acne in respect of the present invention. Lastly, the composition may simultaneously possess the “dermatitis removal function”, “acne germ removal function”, “skin parakeratosis suppression function”, and “sebum supersecretion suppression function”.

### TECHNICAL FIELD

The present invention relates to a composition for treating skin diseases, for example acne, and more particularly, to a composition for acne (formally known as “acne vulgaris”) treatment, which is useful in collectively combating and/or inhibiting “inflammation/bacteria” as well as other causes of acne, and is optimally stabilized to allow long-term storage. Further, the present invention relates to a method for manufacturing the same.

### BACKGROUND ART

Recently, due to rapidly increasing interest in healthy skin, various studies about skin diseases, in particular, acne have been progressed.

In U.S. Pat. No. 5,019,567 entitled “Benzoyl Peroxide Quaternary Ammonium Lipophilic Salicylate Based Pharmaceutical and Cosmetic Compositions and Their Use Especially in Treatment of Acne”, it is disclosed that acne can be treated using antibacterial action of benzoyl peroxide. U.S. Pat. No. 5,554,654 entitled “Method for Enhancing the Therapeutic Effect of an Anti-Acne Agent”, proposes use of hydroxycarboxylic acid, a conventional anti-wrinkle component for acne treatment.

Another reference, U.S. Pat. No. 5,621,006 entitled “Method for Treating Acne Using Benzilic Acid” describes use of benzilic acid for acne treatment. As another acne treatment, thermal spring water is reported in U.S. Pat. Nos. 5,690,946 and 5,997,885, both entitled “Cosmetic and/or Dermatological Composition Containing Thermal Spring Water or Mineral Water and an Active Agent, in order to Combat Acne or Aging”.

Still another reference, U.S. Pat. No. 5,961,993 entitled “Composition Containing a Non-Photocatalytic Metal Oxide and Tocopherol, Its Use in the Treatment of Acne” describes the use of tocopherol and metal oxide for acne treatment. Non-irritating/anti-bacterial active components are reported in the acne treatment of U.S. Pat. No. 6,168,798 entitled “Non-Irritating Composition for Treating Acne and Other Skin Conditions”.

Still other references describe herbs for acne treatment in U.S. Pat. No. 5,248,503 entitled “Herbal Dietary Supplement” and U.S. Pat. No. 5,869,540 entitled “Herbal Treatments for Improving Skin Appearance”. Plant extracts are reported in the acne treatments of U.S. Pat. No. 5,716,800 entitled “Anti-Acne Composition Containing a Poria Cocos Wolf Extract” and U.S. Pat. No. 6,183,747 entitled “Use of Plant Momordica Charactia Extracts for Treatment of Acne Acid”.

In a recent study about the cause of acne, it was found that “inflammation of skin”, “proliferation of acne causing germs, such as bacteria”, “abnormal cornification”, and “excessive sebum production” are major causes of acne.

Specifically, acne is not “a simple skin disease” caused by any one factor, but “a complex skin disease” caused by a complex interaction between all the above causes.

Meanwhile, the prior arts mentioned in the previous patents, i.e., U.S. Pat. Nos. 5,019,567; 5,554,654; 5,621,006; 5,690,946; 5,997,885; 5,961,993; 5,248,503; 5,869,540; 5,716,800; 6,183,747, and the like, mainly focused on anti-inflammatory and anti-bacterial action for acne treatment. Accordingly, use of the prior arts proposed in the previous patents did not provide satisfactory results for acne treatment.

In particular, as for the composition proposed in U.S. Pat. No. 6,168,798, due to its severe side effect of producing unnecessary irritation to skin, its direct application on skin for acne treatment is accompanied with several problems.

Meanwhile, herbal functions such as “protection of skin against damaging external agents such as atmospheric pollutants”, “synthesis of collagen protein forming corium”, and “prevention of pigmentation of skin” are found and various studies for applying herbs in real life situations are in progress.

By way of an example of the above results, U.S. Pat. No. 5,248,503 entitled “Herbal Dietary Supplement” describes inhibition and/or treatment of skin acne by taking herbs. For another reference, U.S. Pat. No. 5,869,540 entitled “Herbal Treatments for Improving Skin Appearance” describes improvement of wrinkles or treatment of skin acne using herbs.

Recently, as herb studies progress, it was found that a volatile fragrance component named “a flavor” among various components of herb, is a critical factor in determining herb's effectiveness.

However, the flavor is easily evaporated by external environmental factors due to its high volatility. Accordingly, in order to apply herbs in real life situations, for example, cosmetic products, how to handle the flavor must be first considered.

U.S. Pat. No. 5,240,732 entitled “Plant Extract-Containing Beverage” discloses use of a high molecular weight substance, such as xanthan gum, as one approach for stabilization of a flavor.

However, a high molecular weight substance such as xanthan gum is widely known to be a hydrophilic material constituted of “sugars of high molecular weight”. Accordingly, stabilization of the flavor using xanthan gum is accompanied with many problems.

For another example, U.S. Pat. No. 5,902,622 entitled “Natural Heat Stable Flavorings for Bakery Applications”, describes a method for stabilizing flavors by emulsifying the flavors using an emulsifier prior to the preparation of bread.

However, the above method is applicable to some degree in bakery applications requiring a short preservation period, but is unsuitable for use in other applications requiring a long preservation period, for example, cosmetic applications.

Recently, as cyclodextrin is widely known as a substance for stabilizing a flavor, studies for flavor encapsulation using cyclodextrin are also widely in progress.

For example, U.S. Pat. No. 5,965,404 entitled “Method for Introducing Nucleic Acids into Higher Eukaryotic Cells” describes a method for protecting a flavor with cyclodextrin in cosmetics for removing odor, wrinkles, and the like. For another example, use of a flavor and cyclodextrin as odor-stabilizing components is reported in U.S. Pat. Nos. 5,942,217; 5,955,093; 6,033,679 and 6,106,738, all entitled “Uncomplexed Cyclodextrin Compositions for Odor Control”.

However, the cyclodextrin has very low solubility in aqueous phase. Accordingly, it is suitable in stabilizing a minor amount of effective ingredient, such as odor, but it is not suitable for use in products combined with other various ingredients, such as cosmetic products.

In addition, the cyclodextrin of the previous patents does not have a molecular structure capable of preventing a flavor from being released into the external environment. As a result, the flavor cannot but be lost to the external environment. Accordingly, the cyclodextrin is not suitable for optimizing the stability of the flavor to desired level.

Therefore, the present invention has been made in view of the above problems, and it is an object of the present invention to provide a composition for acne treatment, which is useful in collectively combating and/or inhibiting “inflammation/bacteria” as well as other causes of acne.

It is another object of the present invention to provide a composition for acne treatment, which is stabilized with hydroxy propyl beta cyclodextrin and silicon polymer.

It is yet another object of the present invention to provide a product for acne treatment, possessing simultaneously such functions as “alleviation of inflammation of skin”, “inhibition of proliferation of acne causing germs”, “inhibition of abnormal cornification”, and “inhibition of excessive sebum production”.

Other objects and advantages of the present invention will be more apparent from a careful reading of the specification below.

### DISCLOSURE OF THE INVENTION

In accordance with the present invention, the above and other objects can be accomplished by the provision of a composition for acne treatment, comprising therapeutically effective ingredients, i.e., 0.01 to 20 parts by weight of herbal extract containing flavors therein, 0.01 to 20 parts by weight of glycyrrhiza extract, 0.01 to 20 parts by weight of phellodendron amurense extract, and 0.001 to 1 parts by weight of salicylic acid.

Preferably, the herbal extract may be burdock extract.

### BEST MODE FOR CARRYING OUT THE INVENTION

In the above composition for acne treatment, the burdock extract serves to inhibit “excessive sebum production”, a cause of acne. Generally, excessive sebum production is known to be induced by an enzyme called 5-α-reductase, which acts on sebaceous gland of skin to produce more sebum. The burdock extract inhibits the activity of 5-α-reductase using tannin acid and flavor contained therein, thereby controlling excessive sebum production.

The glycyrrhiza extract, which is mixed with the burdock extract, plays an important role in alleviating “inflammation of skin”, another cause of acne. It contains “glycyrrhizin acid”, which has an anti-inflammatory activity, as a major component.

According to Korean Patent Application Laid-Open No. 2000-49485 entitled “Method for Producing High Purity Glycyrrhiza Extract” and Korean Patent Application Laid-Open No. 2001-10664 entitled “Antihistamnine Plaster Composition and System for Oral Administration”, glycyrrhizin acid is reported to have excellent anti-inflammatory action. In the present invention, the glycyrrhiza extract, which contains the glycyrrhizin acid as a major component, is combined or mixed with the burdock extract to give a composition for acne treatment. Accordingly, the composition of the present invention retains excellent “anti-inflammatory activity” as well as “excessive sebum production inhibitory activity”.

The phellodendron amurense extract, which is mixed with the above burdock extract and glycyrrhiza extract, plays an important role in inhibiting “proliferation of acne causing germs”, another cause of acne.

The phellodendron amurense extract is reported to have excellent “skin compatibility” in Korean Patent Application Laid-Open No. 1999-75521 entitled “Cosmetics Containing Crude Drug Extract for Improving Wrinkles”, Korean Patent Application Laid-Open No. 2001-10368 entitled “Cosmetic Composition for Controlling Sebum in Skin”, and Korean Patent Application Laid-Open No. 2001-18655 entitled “Cosmetic Composition Containing Rhubarb Root Extract”. Furthermore, the phellodendron amurense extract is reported to retain excellent “antibacterial activity” in U.S. Pat. No. 5,080,900 entitled “Treatment of Skin Ulcers with an Aqueous Extract of Oak Bark Ash”. In the present invention, the phellodendron amurense extract is combined or mixed with the burdock extract and glycyrrhiza extract to give a composition for acne treatment, thereby preventing acne causing germs from proliferating in hair follicles of skin. Accordingly, the composition of the present invention retains excellent “acne causing germs proliferation inhibitory activity”, along with “excessive sebum production inhibitory activity” and “anti-inflammatory activity”.

Then, the salicylic acid of the present invention, which is mixed with the above burdock extract, glycyrrhiza extract and phellodendron amurense extract, plays an important role in inhibiting abnormal cornification, another cause of acne. It generally contains beta-hydroxy acid as a major component. It is reported to have excellent keratin softening activity and wrinkle removal activity in Korean Patent Application Laid-Open No. 1998-43225 entitled “Antifungal Cosmetic Soap Composition”, and Korean Patent Application Laid-Open No. 2001-11241 entitled “Skin Protective Cream Composition”. In the present invention, the salicylic acid is combined or mixed with the above three extracts to give a composition for acne treatment. Accordingly, the composition of the present invention retains excellent “abnormal cornification inhibitory activity”, along with “excessive sebum production inhibitory activity”, “anti-inflammatory activity” and “acne causing germs proliferation inhibitory activity”.

In summary, according to the present invention, the burdock extract, glycyrrhiza extract, phellodendron amurense extract and salicylic acid are mixed in therapeutically effective amounts to give a composition for acne treatment, which is useful in collectively combating and/or inhibiting “inflammation/bacteria” as well as other causes of acne. Accordingly, products prepared using the composition of the present invention retain simultaneously such activities as “alleviation of inflammation of skin”, “inhibition of proliferation of acne causing germs”, “inhibition of abnormal cornification”, and “inhibition of excessive sebum production”.

The composition for acne treatment according to the present invention further comprises 0.01 to 20 parts by weight of hydroxy propyl beta cyclodextrin, and 0.01 to 30 parts by weight of silicon polymer.

Preferably, the hydroxy propyl beta cyclodextrin is added to capture the herbal extract, and the silicon polymer is added to coat the outside of the hydroxy propyl beta cyclodextrin.

Accordingly, the composition of the present invention contains a dual structure, in which the herbal extract is captured by the hydroxy propyl beta cyclodextrin, the outside of which is in turn coated with the silicon polymer.

The hydrophobic moiety, i.e., lipophilic moiety of the hydroxy propyl beta cyclodextrin used in the present invention is oriented towards the inside. The hydrophilic moiety comprising hydrophilic group is positioned toward the outside.

Meanwhile, the silicon polymer used in the present invention is hydrophobic, i.e., lipophilic, as a whole. It contains such siloxane bonds as “Si—O—Si” as a main frame, and has a molecular weight of 300 to 200,000.

The said mixture of the herbal extract, hydroxy propyl beta cyclodextrin, and silicon polymer further comprises 0.01 to 10 parts by weight of antioxidant such as hypotaurine, vitamin E, and super oxide dismutase along with an emulsifier and an oil component.

The antioxidant serves to forestall oxidation of a flavor, an effective component contained in the herbal extract, caused by dissolved oxygen present in each of the components mentioned previously, atmospheric oxygen, and the like.

In summary, as mentioned above, the herbal extract with flavors is captured in the inside of the hydroxy propyl beta cyclodextrin, thereby minimizing contact of the flavors contained in the herbal extract with external substances. As a result, the flavors can be stored in an optimally stable state.

Furthermore, the flavors are captured in the hydroxy propyl beta cyclodextrin, the outside of which is coated with the silicon polymer, to give a dual structure. As a result, oxidation of the flavors contained in the herbal extract by external oxidants is minimized and therefore the flavors can be kept in an optimally stable state.

Accordingly, oxidation of the flavors contained in the herbal extract is also inhibited by the silicon polymer, in addition to the above antioxidant.

As mentioned previously, the lipophilic moiety of the hydroxy propyl beta cyclodextrin used in the present invention is oriented towards the inside and the hydrophilic moiety is positioned toward the outside. While, the epidermis of skin consists of the lipophilic system and the corium consists of the hydrophilic system. Accordingly, the composition of the present invention retains excellent skin absorption ability. After skin absorption, the composition has a mild action, thereby unexpected skin irritation being avoided.

Hereinafter, a method for producing the composition for acne treatment according to the present invention will be described in detail.

First, 0.01 to 20 parts by weight of burdock extract with flavors, 0.01 to 20 parts by weight of glycyrrhiza extract, and 0.01 to 20 parts by weight of phellodendron amurense extract are mixed in 10 parts by weight of purified water.

If the content of the burdock extract is less than 0.01 parts by weight, the addition of herb is insignificant. While, if the content of the burdock extract exceeds 20 parts by weight, there are problems in that flavor of the final product is too strong and no additional effect is seen, thereby causing waste of material. Therefore, it is preferable to limit the content of the burdock extract to “0.01 to 20 parts by weight”.

If the content of the glycyrrhiza extract is less than 0.01 parts by weight, the addition of the glycyrrhiza extract is insignificant. While, if the content of the glycyrrhiza extract exceeds 20 parts by weight, there are problems in that flavor of the final product is too strong and no additional effect is seen, thereby causing waste of material. Therefore, it is preferable to limit the content of the glycyrrhiza extract to “0.01 to 20 parts by weight”.

If the content of the phellodendron amurense extract is less than 0.01 parts by weight, the addition of the phellodendron amurense extract is insignificant. While, if the content of the phellodendron amurense extract exceeds 20 parts by weight, there are problems in that flavor of the final product is too strong and no additional effect is seen, thereby causing waste of material. Therefore, it is preferable to limit the content of the phellodendron amurense extract to “0.01 to 20 parts by weight”.

Then, in order to improve homogeneity in the above mixture, the mixture is agitated in an agitator, for example, at 600 rpm, for 8 to 12 minutes. After the mixing and agitating procedures, a first mixing intermediate is formed.

Apart from the formation of the first mixing intermediate, 0.001 to 1 parts by weight, preferably 0.01 to 0.5 parts by weight of salicylic acid is mixed with 1 to 50 parts by weight, preferably 5 to 30 parts by weight of alcohol, to give a second mixing intermediate.

If the content of the salicylic acid is less than 0.001 parts by weight, the addition of the salicylic acid is insignificant. While, if it exceeds 1 part by weight, there is a problem in that unnecessary skin irritation is caused. Therefore, it is preferable to limit the content of the salicylic acid to “0.001 to 1 parts by weight”.

If the content of the alcohol is less than 1 part by weight, there are problems in that the salicylic acid is not sufficiently dissolved and even if dissolved in the alcohol, unnecessary precipitates is formed. While, if it exceeds. 50 parts by weight, unnecessary skin irritation can be caused. Therefore, it is preferable to limit the content of the alcohol to “1 to 50 parts by weight”.

After completion of the formation of the first and second mixing intermediates, the first and second mixing intermediates are homogeneously mixed. To the mixing intermediates mixture, 0.01 to 20 parts by weight, preferably 0.1 to 5 parts by weight of hydroxy propyl beta cyclodextrin is added. In this case, the hydroxy propyl beta cyclodextrin is added to an extent that its molar content is 10% to 20% more than that of the herbal extract.

If the content of the hydroxy propyl beta cyclodextrin is less than 0.01 parts by weight, the hydroxy propyl beta cyclodextrin cannot suitably capture the effective component, i.e., flavors of the herbal extract. While, if it exceeds 20 parts by weight, the total cost of material increases. Therefore, it is preferable to limit the content of the hydroxy propyl beta cyclodextrin to “0.01 to 20 parts by weight”.

In order to sufficiently capture the flavors contained in the herbal extract in the inside of the hydroxy propyl beta cyclodextrin in the mixture of the herbal extract and the hydroxy propyl beta cyclodextrin, the herbal extract and the hydroxy propyl beta cyclodextrin are agitated in an agitator at 600 rpm, for 30 to 60 minutes. It may be understood that even without the agitating procedure, in case of briefly mixing and then letting the herbal extract and hydroxy propyl beta cyclodextrin stand, for example, for 30 to 60 minutes, the flavors contained in the herbal extract is spontaneously captured in the inside of the hydroxy propyl beta cyclodextrin.

During the mixing and agitating procedures, in order to further greatly enhance the stability of the flavors, 0.01 to 10 parts by weight, preferably 0.1 to 5 parts by weight of antioxidant-such as hypotaurine, vitamin E, and super oxide dismutase is added. If the content of the antioxidant is less than 0.01 parts by weight, the stabilization effect of the flavors is insignificant, while if it exceeds 10 parts by weight, no additional effect is seen, thereby causing waste of material. Therefore, it is preferable to limit the content of the antioxidant to “0.01 to 10 parts by weight”.

Upon completion of the mixing and agitating procedures after 25 to 30 minutes, the herbal extract is captured in the inside of the hydroxy propyl beta cyclodextrin.

In this state, 0.01 to 30 parts by weight of silicon polymer is added. Upon completion of the addition procedure, the outside of the hydroxy propyl beta cyclodextrin capturing the flavors of the herbal extract is coated with the silicon polymer.

After completion of forming the mixture of the first and second mixing intermediates, the mixed resultant is heated at a temperature of 75° C. to 85° C.

In the course of the heating procedure, humectants such as vegetable oil, cetostearyl alcohol, glycerol stearate, polyoxyethylene, and stearate are added in the above mixed resultant. As a result, the final composition for acne treatment according to the present invention can retain a wetting ability for a long time.

After completion of the heating procedure, the heated resultant is cooled to a temperature of 35° C. to 45° C. Then, pigments, antiseptics and perfumes are further added, followed by agitating, to give the final composition for acne treatment according to the present invention.

### EXAMPLES

Hereinafter, the present invention will be described in detail by way of illustrative examples and comparative examples.

First, in example 1, as shown in Table 1 below, 0.1 parts by weight of burdock extract, 0.1 parts by weight of glycyrrhiza extract, and 0.1 parts by weight of phellodendron amurense extract were mixed and agitated in 10 parts by weight of purified water at 600 rpm for 10 minutes to give a first mixing intermediate. Apart from the first mixing intermediate, 5 parts by weight of alcohol and 0.1 parts by weight of salicylic acid were mixed to give a second mixing intermediate.

TABLE 1

| Composition | | | | | | |
|---|---|---|---|---|---|---|
| (part by weight) | Example 1 | Example 2 | Comp. 1 | Comp. 2 | Comp. 3 | Comp. 4 |
| Burdock extract | 0.1 | 0.1 | 1 | | | |
| Glycyrrhiza | 0.1 | 1 | | | | 1 |
| extract | | | | | | |
| Phellodendron | 0.1 | 1 | | | 1 | |
| amurense | | | | | | |
| extract | | | | | | |
| Salicylic acid | 0.1 | 1 | | 1 | | |
| Purified water | 10 | 10 | 10 | | 10 | 10 |
| Alcohol | 5 | 5 | | 5 | | |
| Vegetable oil | 10 | 10 | 10 | 10 | 10 | 10 |
| Cetostearyl | 2 | 2 | 2 | 2 | 2 | 2 |
| alcohol | | | | | | |
| Glycerol | 2 | 2 | 2 | 2 | 2 | 2 |
| stearate | | | | | | |
| Polyoxyethylene | 100 | 100 | 100 | 100 | 100 | 100 |
| Stearate | 1 | 1 | 1 | 1 | 1 | 1 |
| Perfume, | Typical | Typical | Typical | Typical | Typical | Typical |
| pigment | | | | | | |

Then, the first and the second mixing intermediates were homogeneously mixed while agitating and then heated to 80° C. In the course of the heating procedure, 10 parts by weight of vegetable oil, 2 parts by weight of cetostearyl alcohol, 2 parts by weight of glycerol stearate, 100 parts by weight of polyoxyethylene, and 1 part by weight of stearate were added.

After completion of the above procedures, the mixture was cooled to 40° C., followed by addition of a pigment and a perfume, to give a final composition for acne treatment.

In example 2, a final composition for acne treatment was prepared by the same procedure as the example 1 except for using 1 part by weight of burdock extract, 1 part by weight of glycyrrhiza extract, 1 part by weight of phellodendron amurense extract and 1 part by weight of salicylic acid.

In comparative example 1, as shown in Table 1 above, a final composition for acne treatment was prepared by the same procedure as the example 1 except for using 1 part by weight of burdock extract, without using glycyrrhiza extract, phellodendron amurense extract, and salicylic acid.

In comparative example 2, a final composition for acne treatment was prepared by the same procedure as the example 1 except for using 1 part by weight of salicylic acid, without using burdock extract, glycyrrhiza extract, and phellodendron amurense extract.

In comparative example 3, a final composition for acne treatment was prepared by the same procedure as the example 1 except for using 1 part by weight of phellodendron amurense extract, without using burdock extract, glycyrrhiza extract, and salicylic acid.

In comparative example 4, a final composition for acne treatment was prepared by the same procedure as the example 1 except for using 1 part by weight of glycyrrhiza extract, without using burdock extract, phellodendron amurense extract, and salicylic acid.

Hereinafter, based on the test results of the final compositions for acne treatment prepared by “examples 1 and 2” and “comparative examples 1 to 4”, the acne therapeutic effect of the present invention will be described in detail.

TABLE 2

| | Example 1 | Example 2 |
|---|---|---|
| Minimum inhibitory concentration | 750 ppm | 700 ppm |

The compositions for acne treatment prepared by examples 1 and 2 were each filter sterilized to appropriate concentration level and then 1% of activated test bacteria were inoculated into each of the compositions. Then, each of the compositions for acne treatment was cultured anaerobically at a temperature of 37° C. for 48 hours and then the minimum inhibitory concentration (MIC) of each of the compositions to acne causing test bacteria was measured. Based on the obtained results, bacteriostatic activity was evaluated for each of the compositions for acne treatment of the present invention.

As shown in Table 2 above, each of the compositions for acne treatment prepared by examples 1 and 2 inhibited proliferation of acne causing test bacteria in minimum inhibitory concentration of 750 and 700 ppm, respectively. It can be seen from the above result that the composition for acne treatment of the present invention retains excellent “acne causing bacteria proliferation inhibitory activity”.

The present inventors tested acne alleviation degree of the composition for acne treatment according to the present invention.

First, the present inventors selected 20 female persons between the ages of 15 and 20 years, who each bad acne on her face, as a test group. The composition for acne treatment prepared in example 2 was applied to each of the subjects twice a day for 4 weeks.

TABLE 3

| Section | Example 2 | Comparative 1 | Comparative 2 | Comparative 3 | Comparative 4 |
|---|---|---|---|---|---|
| Number of | 8 | 3 | 1 | 0 | 0 |
| persons with | | | | | |
| significant | | | | | |
| improvement | | | | | |
| Number of | 12 | 7 | 6 | 2 | 3 |
| persons with | | | | | |
| moderate and | | | | | |
| slight | | | | | |
| improvement | | | | | |
| Number of | 0 | 10 | 12 | 18 | 17 |
| persons with no | | | | | |
| improvement | | | | | |

In order to accurately compare the acne therapeutic effect of the composition of the present invention with those of the comparative examples, the composition for acne treatment prepared in example 2 was applied on one cheek of each of the subjects and the compositions for acne treatment prepared in comparative examples 1 to 4 were applied on the other cheek of each of the subjects.

After 4 weeks, the present inventors examined degree of alleviation of acne with the unaided eye.

According to the test results as shown in Table 3 above, for all 20 subjects, which had been applied with the composition for acne treatment prepared in example 2, acne on their cheeks was remarkably alleviated. While, for the compositions for acne treatment prepared in comparative examples 1 to 4, acne was alleviated in only 10, 7, 2 and 3 persons, respectively.

It will be understood that the above results support the fact that the composition for acne treatment prepared according to the present invention retains simultaneously such activities as “alleviation of inflammation of skin”, “inhibition of proliferation of acne causing germs”, “inhibition of abnormal cornification”, and “inhibition of excessive sebum production”.

The inventors investigated whether an acne therapeutic effect of the composition according to the present invention is attenuated according to storage time, using the examples and comparative examples shown in Table 4 below.

Because the preparation procedure of each of the examples and comparative examples in Table 4 can be sufficiently deduced from that presented in Table 1, detailed description thereof will be omitted.

As shown in Table 5 below, the inventors stored each of the compositions for acne treatment prepared in “examples 3 to 5” and “comparative examples 5 to 7” “at room temperature of 25° C., for 2 months”, “at high temperature of 110° C. for 2 months”, and “at high temperature of 110° C. for 4 months”, and then measured variations in flavor content in each of the compositions. Based on the obtained results, they evaluated the stability to temperature and light of flavors in each of the compositions.

The compositions for acne treatment prepared in examples 3 to 5 exhibited more than 95% flavor retention rate after storage in each of tested conditions, compared with the compositions for acne treatment prepared in comparative examples 5 to 7. Even after storage at high temperature of 110° C. for 4 months, the compositions for acne treatment prepared in examples 3 to 5 maintained excellent “flavor stability”.

TABLE 4

| Composition | | | | | | |
|---|---|---|---|---|---|---|
| (Part by weight) | Example 3 | Example 4 | Example 5 | Comp. 5 | Comp. 6 | Comp. 7 |
| Composition of | same | Same | same | same | same | Same |
| example 2 | | | | | | |
| Hydroxy propyl beta | 1 | 30 | 1 | | 2 | |
| cyclodextrin | | | | | | |
| Hypotaurine | 0.10 | 0.10 | | | | |
| Poly silicon-11 | 20 | 20 | 20 | | | |

TABLE 5

| Storage | Flavor retention rate (%) | | | | | |
|---|---|---|---|---|---|---|
| condition | Example 3 | Example 4 | Example 5 | Comp. 5 | Comp. 6 | Comp. 7 |
| Room | 98 | 99 | 99 | 91 | 97 | 95 |
| temperature, | | | | | | |
| 2 months | | | | | | |
| 110° C., | 98 | 98 | 98 | 44 | 89 | 75 |
| 2 months | | | | | | |
| 100° C., | 92 | 97 | 94 | 27 | 75 | 64 |
| 4 months | | | | | | |

The present inventors evaluated skin absorption ability of each of the compositions prepared in “examples 3 to 5” and “comparative examples 5 to 7”, according to the skin absorption amount measurement procedure described later.

First, the present inventors cut swine skin, which is generally regarded as the best model for human skin, in a dimension of 20 cm×20 cm to obtain a series of swine skin samples. The swine skin samples were refrigerated at −70° C. The refrigerated samples were cut into squares of 4.5 cm×4.5 cm, and then cold stored in physiological saline.

After a designated time intervals, the samples were cut in a thickness of 1.3 mm. Then, the samples were immobilized at 37° C., for example, in the device effective for skin absorption as disclosed in Christopher L. Gummer, “The In Vitro Evaluation of Transdermal Delivery In Transdermal Drug Delivery”, 1989, pp. 177-196. Then, 1 g of each of the compositions prepared in “examples 3 to 5” and “comparative examples 5 to 7” was taken and applied on the swine skin sample.

500 μl of specimen was taken from each of the above swine skin samples at 6-hour intervals and then skin absorption amount analysis in high performance liquid chromatography (HPLC) was conducted.

According to results as shown in Table 6 below, the compositions prepared in examples 3 to 5 of the present invention exhibited “shorter delay time”, “higher permeation rate”, and “higher permeation coefficient” than the compositions prepared in comparative examples 5 to 7.

The above results support the fact that the compositions prepared in examples 3 to 5 of the present invention have faster skin absorption rate of flavors and higher oxidation inhibition efficiency of flavors, than the compositions prepared in comparative examples 5 to 7.

TABLE 6

| | Absorption delay | Permeation | Permeation |
|---|---|---|---|
| Section | time(sec) | rate(mm/s) | coefficient |
| Example 3 | 3.2 | 74.65 | 0.88 |
| Example 4 | 3.2 | 73.56 | 0.86 |
| Example 5 | 3.3 | 78.41 | 0.91 |
| Comparative 5 | 4.1 | 57.52 | 0.70 |
| Comparative 6 | 3.4 | 71.42 | 0.81 |
| Comparative 7 | 4.8 | 31.58 | 0.33 |

### INDUSTRIAL APPLICABILITY

As apparent from the above description, the present invention provides a composition useful in collectively combating and/or inhibiting “inflammation/bacteria” as well as other causes of acne, obtained by mixing therapeutically effective amounts of burdock extract, glycyrrhiza extract, phellodendron amurense extract, salicylic acid and the like.

Hydroxy propyl beta cyclodextrin and silicon polymer further are contained in the composition of the present invention in order to maintain stability of flavors according to the variation in time.

The final composition for acne treatment according to the present invention possesses simultaneously such abilities as “alleviation of inflammation of skin”, “inhibition of proliferation of acne causing germs”, “inhibition of abnormal cornification”, and “inhibition of excessive sebum production”.

The composition of the present invention can be formulated into, but not limited to, a variety of products for acne treatment, for example in the form of “a cream”, “an essence”, “a pack” and the like, which are effective for acne treatment.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition for acne treatment comprising: 0.01 to 20 parts by weight of herbal extract with a designated amount of effective component; 0.01 to 20 parts by weight of glycyrrhiza extract; 0.01 to 20 parts by weight of phellodendron amurense extract; 0.001 to 1 parts by weight of salicylic acid; 0.01 to 20 parts by weight of hydroxy propyl beta cyclodextrin; and 0.01 to 30 parts by weight of silicon polymer.

2. The composition as set forth in claim 1, wherein the herbal extract is burdock extract.

3. The composition as set forth in claim 1, wherein the hydroxy propyl beta cyclodextrin is mixed in a manner such that it captures the herbal extract.

4. The composition as set forth in claim 1, wherein the silicon polymer is mixed in a manner such that the outside of the hydroxy propyl beta cyclodextrin is coated with the silicon polymer.

5. The composition as set forth in claim 1, wherein the silicon polymer has a molecular weight of 300 to 200,000.

6. The composition as set forth in claim 1, further comprising 0.01 to 10 parts by weight of antioxidant.

7. The composition as set forth in claim 6, wherein the antioxidant is selected from the group consisting of hypotaurine, vitamin E and super oxide dismutase.

8. A method for preparing a composition for acne treatment, comprising the step of:
mixing homogeneously a first mixing intermediate and a second mixing intermediate, the first mixing intermediate being formed by mixing 0.01 to 20 parts by weight of herbal extract with a designated amount of effective component, 0.1 to 20 parts by weight of glycyrrhiza extract and 0.1 to 20 parts by weight of phellodendron amurense extract in a designated amount of purified water, and the second mixing intermediate being formed by mixing 1 to 50 parts by weight of alcohol and 0.001 to 1 parts by weight of salicylic acid;
heating the homogeneous mixture of the first and second mixing intermediates at a designated temperature; and
cooling the heated mixture to a designated temperature.

9. The method as set forth in claim 8, further comprising the step of:
prior to the heating step,
adding and mixing 0.01 to 20 parts by weight of hydroxy propyl beta cyclodextrin in the homogeneous mixture for a designated time, to capture the herbal extract in the inside of the hydroxy propyl beta cyclodextrin; and
adding 0.1 to 30 parts by weight of silicon polymer to the mixture present in a state of the herbal extract being captured in the hydroxy propyl beta cyclodextrin, to coat the outside of the hydroxy propyl beta cyclodextrin with the silicon polymer.

10. The method as set forth in claim 9, further comprising adding 0.01 to 10 parts by weight of antioxidant, in the course of capturing the herbal extract in the inside of the hydroxy propyl beta cyclodextrin.
